(19) United States
(12) Patent Application Publication
(10) Pub. No.: US 2023/0255962 A1
(43) Pub. Date: Aug. 17, 2023

(54) COMBINATION OF A BTK INHIBITOR AND AN INHIBITOR OF CDK9 TO TREAT CANCER

(71) Applicant: ASTRAZENECA AB, SODERTALJE (SE)

(72) Inventors: SCOTT BOIKO, WILMINGTON, DE (US); JUSTIN CIDADO, WILMINGTON, DE (US); LISA DREW, WILMINGTON, DE (US); THERESA PROIA, WILMINGTON, DE (US); MARYANN SAN MARTIN, WILMINGTON, DE (US)

(21) Appl. No.: 17/950,314

(22) Filed: Sep. 22, 2022

Related U.S. Application Data
(63) Continuation of application No. 16/650,042, filed on Mar. 24, 2020, now abandoned.
(60) Provisional application No. 62/562,806, filed on Sep. 25, 2017.

(51) Int. Cl. A61K 31/4985; A61P 35/00; A61K 31/4025; A61K 31/4433; A61K 31/4439; A61K 31/444; A61K 31/505; A61K 31/519; A61K 31/522; A61K 31/53

(52) U.S. Cl. CPC A61K 31/4985; A61P 35/00; A61K 31/4025; A61K 31/4433; A61K 31/4439; A61K 31/444; A61K 31/505; A61K 31/519; A61K 31/522; A61K 31/53

(57) ABSTRACT

The present invention relates to combination of a BTK inhibitor and an inhibitor of CDK9 for use in the treatment of cancer in a subject. The BTK inhibitor can be acalabrutinib, ibrutinib, or ONO/GS-4059; and the inhibitor of CDK9 can be AZD4573, BAY-1251152, BAY-1143572, CYC065, alvocidib, AT7519, voruciclib, roniciclib, or dinaciclib.

### CROSS-REFERENCE TO RELATED APPLICATIONS

This application is a continuation of U.S. application Ser. No. 16/650,042, filed Mar. 24, 2020, said application Ser. No. 16/650,042 is a U.S. National Stage application of International Application No. PCT/IB2018/057374 filed on Sep. 24, 2018, said International Application No. PCT/IB2018/057374 claims benefit under 35 U.S.C. § 119(e) of the U.S. Provisional Application No. 62/562,806, filed Sep. 25, 2017. Each of the above listed applications is incorporated by reference herein in its entirety for all purposes.

### BACKGROUND

Cyclin-dependent protein kinases (CDKs) represent a family of serine/threonine protein kinases that become active upon binding to a cyclin regulatory partner. CDK/cyclin complexes were first identified as regulators of cell cycle progression. CDK/cyclin complexes have also been implicated in transcription and mRNA processing. CDK9/PTEFb (positive transcription elongation factor b) phosphorylates the carboxyl-terminal domain (CTD) of the large subunit of RNA polymerase II (RNAP II), predominantly at Ser-2, regulating elongation of transcription. Inhibition of CDK9 and transcriptional repression results in the rapid depletion of short lived mRNA transcripts and associated proteins, including Mcl1 and c-myc, leading to induction of apoptosis in tumor cells that are hyper-dependent on these survival proteins.

Bruton tyrosine kinase (BTK), a member of the TEC family of kinases, is an important component in the B-cell receptor (BCR) signaling pathway, where it sits between the BCR and downstream survival signals. BTK is expressed in cells of hematopoietic lineage, except for T cells, and is upregulated in chronic lymphocytic leukemia (CLL) cells relative to normal B cells. BTK is also essential for proliferation and survival of some of B-cell malignancies. In particular, knockdown of BTK induces tumor cell death in primary CLL cells and lymphoma cell lines that are dependent on BCR signaling. Furthermore, genetic ablation of BTK inhibits disease progression in mouse models of CLL, indicating its continued importance for B-cell malignancies.

### SUMMARY

The present invention relates to combination treatments for use in the treatment of certain diseases, such as cancer.

In one aspect, a combination of a BTK inhibitor and an inhibitor of CDK9 for use in the treatment of cancer in a subject. The BTK inhibitor can be administered to the subject before the inhibitor of CDK9 is administered to the subject. The BTK inhibitor can be acalabrutinib (also referred to as ACP-196), ibrutinib, or ONO/GS-4059. The BTK inhibitor can be acalabrutinib. The inhibitor of CDK9 can be AZD4573 or dinaciclib. The inhibitor of CDK9 can be AZD4573.

The cancer can be selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In another aspect, a combination of acalabrutinib and AZD4573 can be used in the treatment of cancer in a subject, wherein the acalabrutinib is to be administered to the subject before the AZD4573 is administered to the subject, and wherein the cancer is selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In another aspect, a method of treating cancer in a subject can includes: administering an effective amount of a BTK inhibitor to the subject; and administering an effective amount of an inhibitor of CDK9 to the subject.

The BTK inhibitor can be administered to the subject before the inhibitor of CDK9 is administered to the subject. The inhibitor of CDK9 can be a selective inhibitor of CDK9. The BTK inhibitor can be acalabrutinib, ibrutinib, or ONO/GS-4059. The BTK inhibitor can be acalabrutinib. The inhibitor of CDK9 can be AZD4573 or dinaciclib. The inhibitor of CDK9 can be AZD4573. The cancer can be selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In another aspect, a method of treating cancer in a subject can include: administering an effective amount of acalabrutinib to the subject; and administering an effective amount of AZD4573 to the subject; wherein the acalabrutinib is administered to the subject before the AZD4573 is administered to the subject; and wherein the cancer is selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In another aspect, a kit can include: a first pharmaceutical composition comprising a BTK inhibitor and a pharmaceutically acceptable carrier; and a second pharmaceutical composition comprising an inhibitor of CDK9 and a pharmaceutically acceptable carrier. The BTK inhibitor can be acalabrutinib, ibrutinib, or ONO/GS-4059. The inhibitor of CDK9 can be AZD4573 or dinaciclib. The BTK inhibitor can be acalabrutinib. The inhibitor of CDK9 can be AZD4573. The BTK inhibitor can be acalabrutinib, and the inhibitor of CDK9 can be AZD4573.

Other features, objects, and advantages of the invention will be apparent from the description and drawings, and from the claims.

### BRIEF DESCRIPTION OF THE DRAWINGS

FIG. 1 shows the effect of acalabrutinib treatment on three different DLBCL cell lines.

FIG. 2 illustrates the effect of a combination of AZD4573 with acalabrutinib pre-treatment BTK inhibitor-sensitive cell lines (OCILy10 and TMD8) and a BTKi-insensitive cell line (Karpas422).

FIG. 3 illustrates the effect of acalabrutinib, AZD4573, a combination of AZD4573 and acalabrutinib dosed concurrently, and a combination of AZD4573 and acalabrutinib dosed sequentially, on apoptosis in three different DLBCL cell lines.

FIGS. 4a, 4b, and 4c, illustrate the effects of a combination treatment of acalabrutinib and AZD4573 on in vivo models of DLBCL.

FIGS. 5a and 5b, illustrate the effects of a combination treatment of acalabrutinib and AZD4573 on in vivo models of DLBCL.

### DETAILED DESCRIPTION

Disclosed herein are combination treatments useful for the treatment of proliferative diseases, such as cancer. In particular the combination treatments are of use in the treatment of proliferative disease such as cancer including, but not limited to, diffuse large B-cell lymphoma (DLBCL), mantle cell lymphoma (MCL), chronic lymphocytic leukemia (CLL), small chronic lymphocytic leukemia (SLL), Waldentröm's macroglobulinemia, marginal zone lymphoma, chronic graft versus host disease, follicular lymphoma, and acute lymphoblastic leukemia (ALL), and other BTK-sensitive cancers.

A combination treatment using more than one agent can provide improved effectiveness relative to treatment with either agent alone. (Although this specification frequently discusses two-agent combinations, it is recognized that combinations with three or more agents are possible.)

The term “combination” can refer to simultaneous, separate, or sequential administration of two or more agents. In one aspect, “combination” can refer to simultaneous administration (e.g., administration of both agents in a single dosage form). In another aspect, “combination” refers to separate administration (e.g., administration of both agents in separate dosage forms, but at substantially the same time). In a further aspect of the invention “combination” refers to sequential administration (e.g., where a first agent is administered, followed by a delay, followed by administration of a second or further agent). Where the administration is sequential or separate, the delay in administering the later component should be neither too long nor too short, so as not to lose the benefit of the combination.

As used herein, the term “an inhibitor of CDK9” refers to a compound that can inhibit CDK9, and, optionally, can inhibit one or more other CDKs. A compound that inhibits one or more other CDKs in addition to CDK9 is a non-selective inhibitor of CDK9, even if the primary target of the compound is not CDK9. For example, dinaciclib inhibits multiple CDKs, including CDK9. Thus, dinaciclib is a non-selective inhibitor of CDK9, as the term is used herein. A selective inhibitor of CDK9 is a compound that inhibits CDK9 and has little or no inhibitory activity toward other CDKs. Thus, “an inhibitor of CDK9” as used herein includes both non-selective and selective inhibitors of CDK9.

The terms “treat,” “treating,” and “treatment” refer to at least partially alleviating, inhibiting, preventing and/or ameliorating a condition, disorder, or disease, such as cancer. The terms “treatment of cancer” or “treatment of cancer cells” include both in vitro and in vivo treatments, including in warm-blooded animals such as humans. The effectiveness of treatment of cancer cells can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival. Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena. Other assessments of effectiveness are known to those of skill in the art.

A combination of a BTK inhibitor and an inhibitor of CDK9 can be more effective than either alone in treatment of cancer cells (whether in vitro or in vivo). In particular, treatment of BTK-responsive cancer cells can sensitize those cells to treatment with an inhibitor of CDK9. In this regard, effectiveness of a combination treatment can be assessed in one or more of the ways described above. Accordingly, in some embodiments, a combination of a BTK inhibitor and an inhibitor of CDK9, when used in the treatment of cancer cells, can be more effective than either agent alone. The combination treatment can be more effective in one or more of: promoting cancer cell death; inhibiting cancer growth (e.g., inhibiting an increase in tumor volume); and duration of response.

BTK inhibitors include, for example, acalabrutinib, ibrutinib, and ONO/GS-4059.

Acalabrutinib, also referred to as ACP-196 or (S)-4-(8-amino-3-(1-but-2-ynoylpyrrolidin-2-yl)imidazo[1,5-a]pyrazin-1-yl)-N-(pyridin-2-yl)benzamide, has the formula:

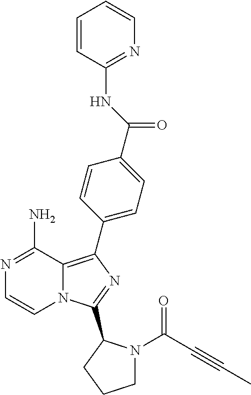 [SMILES: CC#CC(=O)N1CCC[C@H]1c1nc(-c2ccc(C(=O)Nc3ccccn3)cc2)c2c(N)nccn12]

Methods of making acalabrutinib are described in, for example, WO 2013/010868 which is incorporated by reference in its entirety.

Inhibitors of CDK9 include, for example, AZD4573, BAY-1251152, BAY-1143572, CYC065, alvocidib, AT7519, voruciclib, roniciclib, and dinaciclib. Selective inhibitors of CDK9 include AZD4573, BAY-1251152, and BAY-1143572. Non-selective inhibitors of CDK9 include CYC065, alvocidib, AT7519, voruciclib, roniciclib, and dinaciclib.

AZD4573, a selective CDK9 inhibitor, also referred to as (1S,3R)-3-acetamido-N-(5-chloro-4-(5,5-dimethyl-5,6-dihydro-4H-pyrrolo[1,2-b]pyrazol-3-yl)pyridin-2-yl)cyclohexanecarboxamide, has the formula:

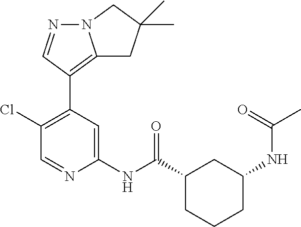 [SMILES: CC(=O)N[C@@H]1CCC[C@H](C(=O)Nc2cc(-c3cnn4c3CC(C)(C)C4)c(Cl)cn2)C1]

Methods of making AZD4573 are described in, for example, WO 2017/001354, which is incorporated by reference in its entirety.

In some embodiments, the combination can include acalabrutinib, ibrutinib, or ONO/GS-4059. In some embodiments, the combination can include AZD4573, BAY-1251152, BAY-1143572, CYC065, alvocidib, AT7519, voruciclib, roniciclib, and dinaciclib. In some embodiments, the combination can include acalabrutinib. In some embodiments, the combination can include AZD4573. In some embodiments, the combination can include acalabrutinib and AZD4573.

In some embodiments, sequential administration of the BTK inhibitor and the inhibitor of CDK9 can have greater effectiveness than simultaneous administration. In some embodiments, the BTK inhibitor can be administered before the inhibitor of CDK9 is administered within a dosage cycle.

In some embodiments, the BTK inhibitor can be administered at least 2 hours, at least 4 hours, at least 6 hours, at least 8 hours, at least 12 hours, at least 16 hours, at least 24 hours, or at least 48 hours before the inhibitor of CDK9 is administered within a dosage cycle.

In some embodiments, the BTK inhibitor can be administered no more than 2 hours, no more than 4 hours, no more than 6 hours, no more than 8 hours, no more than 12 hours, no more than 16 hours, no more than 24 hours, or no more than 48 hours before the inhibitor of CDK9 is administered within a dosage cycle.

In some embodiments, the BTK inhibitor can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before the inhibitor of CDK9 is administered within a dosage cycle.

In some embodiments, administering the BTK inhibitor on an intermittent dosage schedule can have greater effectiveness than a dosing on a continuous dosage schedule. In some embodiments, administering the inhibitor of CDK9 on an intermittent dosage schedule can have greater effectiveness than a dosing on a continuous dosage schedule. In some embodiments, administering both the BTK inhibitor and the inhibitor of CDK9 on an intermittent dosage schedule can have greater effectiveness than a dosing on a continuous dosage schedule.

An intermittent dosage schedule can include dosage holidays. For the purposes of illustration, in a seven-day dosage cycle, an intermittently dosed agent might be given on days one and two, but not given on days three, four, five, six, or seven. The dosage cycle would then repeat. This illustration could be referred to as a 2 on/5 off schedule, where the agent is given for two days followed by a five day holiday.

A continuous dosing schedule, in contrast, includes no holidays during a dosage cycle. Thus, for illustration, in a in a seven-day dosage cycle, an continuously dosed agent would be given on days one, two, three, four, five, six, and seven. The dosage cycle would then repeat.

In some embodiments, the dosage cycle may be from 5 to 30 days, from 7 to 24 days, from 9 to 16 days, or from 13 to 15 days. In some embodiments, the dosage cycle may be 5 days, 7 days, 10 days, 12 days, 14 days, 21 days, or 28 days.

In some embodiments, the BTK inhibitor can be dosed continuously in a dosage cycle.

In some embodiments, the inhibitor of CDK9 can be dosed intermittently in a dosage cycle.

In some embodiments, the BTK inhibitor can be dosed continuously, and the inhibitor of CDK9 can be dosed intermittently, in a dosage cycle.

In some embodiments, the inhibitor of CDK9 can be administered on from 2 to 4 days, and not administered on the remaining days, in a dosage cycle of from 6 to 16 days.

In some embodiments, the inhibitor of CDK9 can be administered on from 2 to 3 consecutive days, and not administered on the remaining days, in a dosage cycle of from 13 to 15 days.

In some embodiments, the inhibitor of CDK9 can be administered on 2 consecutive days, and not administered on the remaining days, in a dosage cycle of 14 days.

In some embodiments, the BTK inhibitor can be administered every day, and the inhibitor of CDK9 administered on only two days, and not administered on the remaining days, in a dosage cycle.

In some embodiments, the BTK inhibitor can be administered every day, and the inhibitor of CDK9 administered on only two consecutive days, and not administered on the remaining days, in a dosage cycle of 14 days.

In some embodiments, acalabrutinib can be dosed continuously, and AZD4573 dosed intermittently, in a dosage cycle.

In some embodiments, acalabrutinib can be administered every day, and AZD4573 administered on only two days, and not administered on the remaining days, in a dosage cycle.

In some embodiments, acalabrutinib can be administered every day, and AZD4573 administered on only two consecutive days, and not administered on the remaining days, in a dosage cycle of 14 days.

In some embodiments, the BTK inhibitor can be dosed continuously, the inhibitor of CDK9 can be dosed intermittently, and the BTK inhibitor can be administered before the inhibitor of CDK9 is administered, in a dosage cycle.

In some embodiments, the inhibitor of CDK9 can be administered on from 2 to 4 days, and not administered on the remaining days, and the BTK inhibitor can be administered before the inhibitor of CDK9 is administered, in a dosage cycle of from 6 to 16 days.

In some embodiments, the inhibitor of CDK9 can be administered on 2 consecutive days, and not administered on the remaining days, and the BTK inhibitor can be administered before the inhibitor of CDK9 is administered, in a dosage cycle of 14 days.

In some embodiments, the BTK inhibitor can be administered every day, and the inhibitor of CDK9 can be administered on only two days, and not administered on the remaining days, and the BTK inhibitor can be administered before the inhibitor of CDK9 is administered, in a dosage cycle.

In some embodiments, the BTK inhibitor can be administered every day, and the inhibitor of CDK9 can be administered on only two consecutive days, and not administered on the remaining days, and the BTK inhibitor can be administered before the inhibitor of CDK9 is administered, in a dosage cycle of 14 days.

In some embodiments, acalabrutinib can be dosed continuously, and AZD4573 can be dosed intermittently, and acalabrutinib can be administered before AZD4573 is administered, in a dosage cycle.

In some embodiments, 100 mg of acalabrutinib can be administered two times each day, and AZD4573 can be dosed intermittently, and acalabrutinib can be administered before AZD4573 is administered, in a dosage cycle.

In some embodiments, acalabrutinib can be administered every day, and AZD4573 can be administered on only two days, and not administered on the remaining days, and acalabrutinib can be administered before AZD4573 is administered, in a dosage cycle.

In some embodiments, acalabrutinib can be administered every day, and AZD4573 administered on only two consecutive days, and not administered on the remaining days, and acalabrutinib can administered before AZD4573 is administered, in a dosage cycle of 14 days.

In some embodiments, 100 mg of acalabrutinib can be administered two times each day, and AZD4573 administered on only two consecutive days, and not administered on the remaining days, and acalabrutinib can administered before AZD4573 is administered, in a dosage cycle of 14 days

In some embodiments, the BTK inhibitor can be dosed continuously, and the inhibitor of CDK9 can be dosed intermittently, and the BTK inhibitor can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before the inhibitor of CDK9 is administered, in a dosage cycle.

In some embodiments, the inhibitor of CDK9 can be administered on from 2 to 4 days, and not administered on the remaining days, and the BTK inhibitor can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before the inhibitor of CDK9 is administered, in a dosage cycle of from 6 to 16 days.

In some embodiments, the inhibitor of CDK9 can be administered on 2 consecutive days, and not administered on the remaining days, and the BTK inhibitor can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before the inhibitor of CDK9 is administered, in a dosage cycle of 14 days.

In some embodiments, the BTK inhibitor can be administered every day, and the inhibitor of CDK9 can be administered on only two days, and not administered on the remaining days, and the BTK inhibitor can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before the inhibitor of CDK9 is administered, in a dosage cycle.

In some embodiments, the BTK inhibitor can be administered every day, and the inhibitor of CDK9 can be administered on only two consecutive days, and not administered on the remaining days, and the BTK inhibitor can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before the inhibitor of CDK9 is administered, in a dosage cycle.

In some embodiments, acalabrutinib can be dosed continuously and AZD4573 can be dosed intermittently, and acalabrutinib can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before AZD4573 is administered, in a dosage cycle.

In some embodiments, 100 mg of acalabrutinib can be administered two times each day, and AZD4573 can be dosed intermittently, and acalabrutinib can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before AZD4573 is administered, in a dosage cycle.

In some embodiments, acalabrutinib can be administered every day, and AZD4573 can be administered on only two days, and not administered on the remaining days, and acalabrutinib can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before AZD4573 is administered, in a dosage cycle.

In some embodiments, acalabrutinib can be administered every day, and AZD4573 can be administered on only two consecutive days, and not administered on the remaining days, and acalabrutinib can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before AZD4573 is administered, in a dosage cycle of 14 days.

In some embodiments, 100 mg of acalabrutinib can be administered two times each day, and AZD4573 can be administered on only two consecutive days, and not administered on the remaining days, and acalabrutinib can be administered from 2 to 4 hours before; from 4 to 6 hours before; from 6 to 8 hours before; from 8 to 12 hours before; from 12 to 16 hours before; from 16 to 24 hours before; from 20 to 28 hours before; or from 24 to 48 hours before AZD4573 is administered, in a dosage cycle of 14 days.

In some embodiments, a BTK inhibitor can be used in the treatment of cancer in a subject, wherein the treatment can include the separate, sequential, or simultaneous administration of the BTK inhibitor and an inhibitor of CDK9 to the subject.

In some embodiments, the treatment can include the administration of the BTK inhibitor to the subject before the administration of the inhibitor of CDK9 to the subject.

In some embodiments, the BTK inhibitor can be acalabrutinib, ibrutinib or ONO/GS-4059. In some embodiments, the BTK inhibitor can be acalabrutinib.

In some embodiments, the inhibitor of CDK9 can be AZD4573, BAY-1251152, BAY-1143572, CYC065, alvocidib, AT7519, voruciclib, roniciclib, and dinaciclib. In some embodiments, the inhibitor of CDK9 can be AZD4573.

In some embodiments, the cancer can be selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In some embodiments, a BTK inhibitor for use in the treatment of cancer in a subject, wherein the treatment can include the separate, sequential, or simultaneous administration of the BTK inhibitor and an inhibitor of CDK9 to the subject; wherein the BTK inhibitor is acalabrutinib; the inhibitor of CDK9 is AZD4573; and the cancer can be selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In some embodiments, an inhibitor of CDK9 can be used for use in the treatment of cancer in a subject, wherein the treatment can include the separate, sequential, or simultaneous administration of the inhibitor of CDK9 and a BTK inhibitor to the subject.

In some embodiments, the treatment can include the administration of the BTK inhibitor to the subject before the administration of the inhibitor of CDK9 to the subject.

In some embodiments, the BTK inhibitor can be acalabrutinib, ibrutinib or ONO/GS-4059. In some embodiments, the BTK inhibitor can be acalabrutinib.

In some embodiments, the inhibitor of CDK9 can be AZD4573, BAY-1251152, BAY-1143572, CYC065, alvocidib, AT7519, voruciclib, roniciclib, and dinaciclib. In some embodiments, the inhibitor of CDK9 can be AZD4573.

In some embodiments, the cancer can be selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

In some embodiments, an inhibitor of CDK9 can be used in the treatment of cancer in a subject, wherein the treatment can include the separate, sequential, or simultaneous administration of the inhibitor of CDK9 and a BTK inhibitor to the subject; wherein the BTK inhibitor is acalabrutinib; the inhibitor of CDK9 is AZD4573; and the cancer can be selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

### EXAMPLES

### Example 1: Acalabrutinib Primes BTK Inhibitor-Sensitive Cancer Cells Lines to Die through Increased Protein Levels of Pro-Apoptotic BH3-Only Proteins

### Method

Two ABC-DLBCL (OCILy10 and TMD8) cell lines and one GCB-DLBCL (Karpas422) cell line were treated with either vehicle (DMSO) or a 10-point, ½ log serially dilution of acalabrutinib for 72 hours with a CellTiter-Glo viability readout. The concentration at which 50% growth is inhibited (GI₅₀) was calculated using GraphPad Prism or GeneData.

OCILy10, TMD8, and Karpas422 were treated with either vehicle or 100 nM acalabrutinib for 2 to 72 hours. Protein lysates were harvested at multiple time points in that interval, protein concentration was determined using the BCA Protein Assay Kit, and Western blots were performed according to standard protocol to assess the effects on Bcl2 family protein levels. Each sample was normalized to vinculin as a loading control, and then protein levels of acalabrutinib-treated samples were calculated relative to those of vehicle-treated samples.

### Discussion

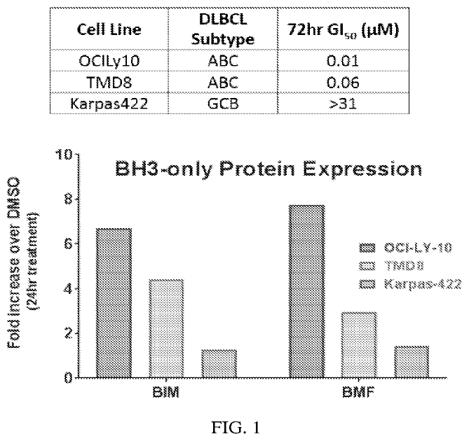

DLBCL cell lines display a differential sensitivity to the BTK inhibitor acalabrutinib. A dose-response of acalabrutinib in three DLBCL cell lines showed that two ABC-DLBCL cell lines, OCILy10 and TMD8, were sensitive to BTK inhibition while the GCB-DLBCL cell line, Karpas422, was not. Each of the three cell lines were then treated with 100 nM acalabrutinib for 24 hours, and BH3-only protein levels were assessed. Pro-apoptotic BH3-only proteins bind to inhibit the anti-apoptotic function of major Bcl-2 family proteins. Results are illustrated in FIG. 1. The levels of pro-apoptotic BH3-only proteins Bim and Bmf were increased in the two acalabrutinib-sensitive DLBCL cell lines relative to the insensitive cell line, priming the cells for apoptosis.

### Example 2: Combination of Acalabrutinib and AZD4573 Leads to Enhanced and Rapid Induction of Cell Death

### Method

Two BTK inhibitor-sensitive ABC-DLBCL cell lines (OCILy10 and TMD8) and one insensitive GCB-DLBCL cell line (Karpas422) were treated for an 8-hour time course with either 100 nM AZD4573 alone or following a 24-hour pre-treatment with 100 nM acalabrutinib (since exposure to acalabrutinib for 24 hours was sufficient to induce maximal BH3-only protein levels). Cells were harvested for protein lysates at varying time points (0, 0.5, 1, 2, 4, and 8 hours) post-AZD4573 treatment, normalized for protein concentration using the BCA Protein Assay Kit, and run for western blots according to standard protocols. To ensure expected target engagement was achieved, the blots were probed for the proximal and distal biomarkers for CDK9 (pSer2-RNAPolII and Mcl-1, respectively) and the biomarker for activated BTK (pBTK). To gauge the time to induction of apoptosis, cleaved caspase-3 was also assessed. A loading control (vinculin) was also utilized for normalization.

### Discussion

Since acalabrutinib increases protein levels of Bim and Bmf in BTK inhibitor-sensitive cell lines, priming the cells for apoptosis, combination with AZD4573, which rapidly depletes Mcl1 levels, was predicted to tilt the balance of pro- and anti-apoptotic Bcl2 family proteins toward cell death. Therefore, two BTK inhibitor-sensitive cell lines and one insensitive cell line were treated with either AZD4573 alone, or following pre-treatment with acalabrutinib.

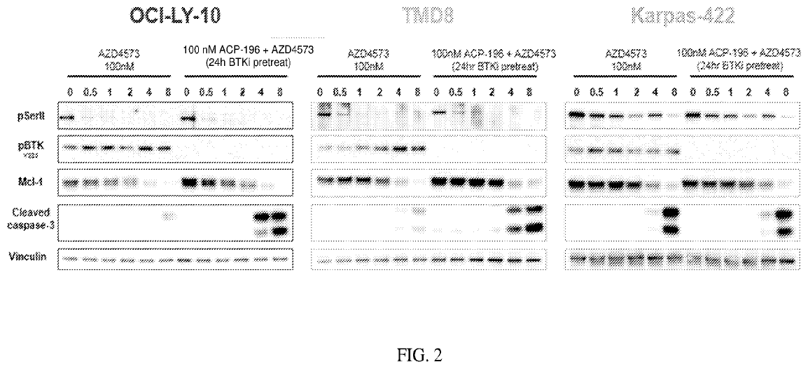

In the two BTK inhibitor-sensitive cell lines (OCILy10 and TMD8), the combination resulted in the robust induction of cleaved caspase by 4 hours, whereas AZD4573 alone resulted in mild caspase activation by 8 hours. In the BTK inhibitor-insensitive cell line (Karpas422), where acalabrutinib did not increase BH3-only protein levels, there was no difference in caspase activation between the single agent or combination samples. See FIG. 2.

### Example 3: Acalabrutinib Pre-Treatment Enhances the Combination Benefit with AZD4573

### Method

Two BTK inhibitor-sensitive ABC-DLBCL cell lines (OCILy10 and TMD8) and one insensitive GCB-DLBCL cell line (Karpas422) were treated with either vehicle (DMSO), 100 nM acalabrutinib, or 100 nM AZD4573 for varying lengths of time as either single agent or in combination. Since exposure of BTKi-sensitive cell lines to acalabrutinib for 24 hours was sufficient to induce maximal BH3-only protein levels relative to vehicle-treated cells (FIG. 1), 24-hour pre-treatment of acalabrutinib was chosen for one of the combination sequences. Therefore, cells were treated with: 32 hours of acalabrutinib alone; 8 hours of AZD4573 alone; 8 hours of AZD4573 and acalabrutinib dosed concurrently; or 32 hours of acalabrutinib with AZD4573 added sequentially for the final 8 of those 32 hours (in other words, 24 hours of acalabrutinib pre-treatment).

The percentage of apoptotic or dead cells was determined by staining the samples with both tetramethylrhodamine, ethyl ester (TMRE) and 4′,6-diamidino-2-phenylindole (DAPI) and then subjecting them to flow cytometry using a BD LSR Fortessa. TMRE is a positively charged dye that accumulates in active mitochondria, and cells undergoing apoptosis experience mitochondrial outter membrane permeabilization (MOMP), which then fails to retain TMRE. Hence, a decrease in TMRE staining is indicative of apoptosis. DAPI is a DNA-binding fluorescent dye that is membrane impermeable in viable cells, so dying cells will exhibit increased DAPI staining. Following appropriate treatments, cells were stained directly with 200 nM TMRE, incubated for 30 minutes at 37° C., resuspended in flow buffer containing DAPI, and subjected to flow cytometry. Flowjo was used to establish appropriate gates according to viability (DAPI) and MOMP (TMRE), and viable cells were defined as those with low DAPI and high TMRE staining.

### Discussion

Acalabrutinib treatment of BTK inhibitor-sensitive cells increased BH3-only proteins levels over time with maximal levels achieved by 24 hours following dosing (FIG. 1). However, it was unclear what length, if any, of acalabrutinib pre-treatment would enhance the combination benefit with AZD4573. In this Example, acalabrutinib was dosed either concurrently or sequentially with AZD4573 and assessed for apoptotic/dead cells following the final 8 hours of AZD4573 treatment.

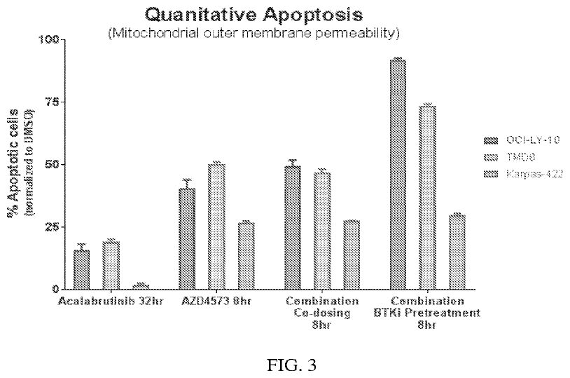

Treatment of the OCI-LY-10 cells with either 32 hours of acalabrutinib alone, or 8 hours of AZD4573 alone, resulted in ˜20% and ˜40% cell death, respectively (FIG. 3). When the inhibitors were dosed concurrently (labelled co-dosing in FIG. 3) for 8 hours, there was a minimal increase in apoptotic cells (˜50%). In contrast, when dosed sequentially with 32 hours of acalabrutinib and AZD4573 for the final 8 hours, almost 90% of the cells were apoptotic. Hence, pre-treatment with acalabrutinib significantly increased the benefit of combination with AZD4573 over concurrent treatment. Similar results for TMD8 cells were observed. In Karpas-422 cells, acalabrutinib alone or in combination had minimal effect, as expected for a BTK-insensitive cell line.

### Example 4: Combination of AZD4573 with Acalabrutinib in BTK Inhibitor-Sensitive In Vivo Models Results in Enhanced Anti-Tumor Activity

### Method

AZD4573 was formulated in dimethylacetamide (DMA)/polyethylene glycol 400 (PEG 400)/1% w/v Tween 80 solution 2/30/68 and dosed at 5 mL/kg, intraperitoneally (ip), BID with a 2 hour split on days 1 and 2 followed by a 5 day dose holiday; i.e., a 2 on/5 off schedule.

Acalabrutinib was formulated in 0.5% hydroxypropyl methyl cellulose/0.2% Tween 80, and dosed twice a day (bid) as an oral (po) administration at a volume of 10 ml/kg at a dose of 12.5 mg/kg. For the OCILy10 model, acalabrutinib was dosed using an 8/16 hr split BID dosing schedule. For TMD8 and Karpas-422 studies, acalabrutinib was dosed using a 12 hr BID split dosing schedule. For all three cell lines, the first dose of AZD4573 was administered 24 hr after the first administration of acalabrutinib.

5×10⁶ OCILy10 tumor cells or 10×10⁶ TMD8 tumor cells were injected subcutaneously in the right flank of C.B.-17 SCID female mice in a volume of 0.1 mL containing 50% matrigel. 10×10⁶ Karpas-422 tumor cells were injected subcutaneously in the right flank of SCID beige female mice in a volume of 0.1 mL containing 50% matrigel. Tumor volumes (measured by caliper), animal body weight, and tumor condition were recorded twice weekly for the duration of the studies. The tumor volume was calculated using the formula: length (mm)×width (mm)²×0.52. For efficacy studies, growth inhibition from the start of treatment was assessed by comparison of the differences in tumor volume between control and treated groups. Dosing began when mean tumor size reached approximately 150-180 mm³. CR=complete response.

### Discussion

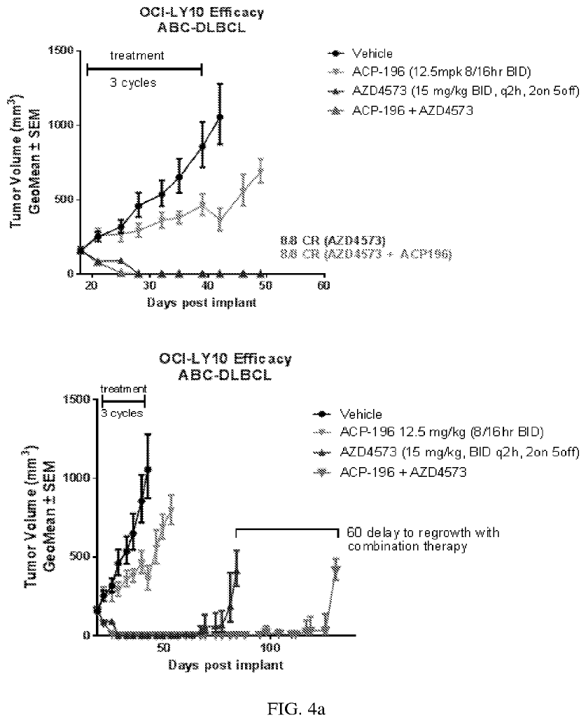

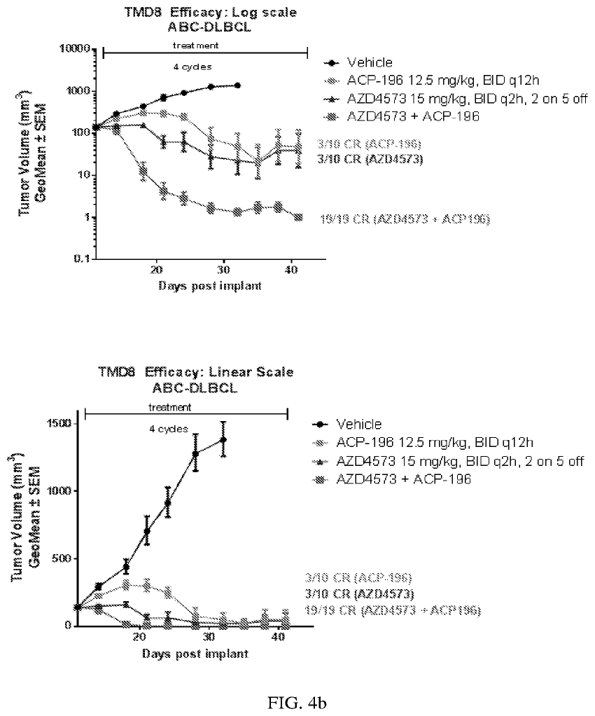

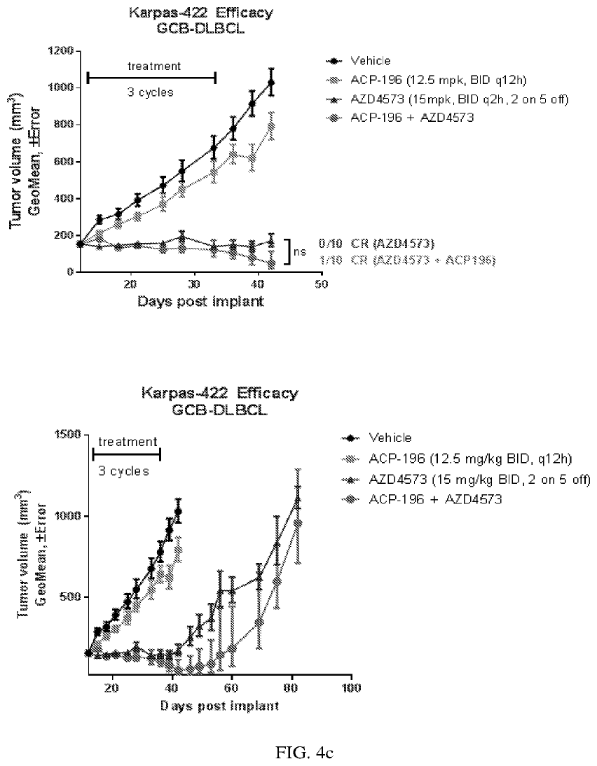

Combining AZD4573 with acalabrutinib in ABC-DLBCL models improved duration of response (OCILy10, FIG. 4a) and lead to complete regressions (TMD8, FIG. 4b). However, no combination benefit was observed in the GBC-DLBCL model Karpas-422 (FIG. 4c). Surprisingly, twice weekly administration of AZD4573 in combination with acalabrutinib yielded a 60 day delay to tumor regrowth in OCILy10 compared with single agent AZD4573 treatment (FIG. 4a, lower panel). In TMD8, twice weekly administration of AZD4573 combined with acalabrutinib produced complete responses in 100% of treated animals (FIG. 4b, label 19/19 CR), which was maintained for 53 days after treatment removal. 18/19 animals remained tumor free out to 61 days past the end of the dosing period (data not shown). In contrast, no combination benefit was observed in the GCB-DLBCL model Karpas-422 following 3 cycles of treatment, as expected for a BTK-insensitive cell line. All therapies were well tolerated whether dosed alone or in combination.

Note that in FIGS. 4a, 4b, and 4c, acalabrutinib is designated as ACP-196.

### Example 5: Alternative BTK Inhibitor Ibrutinib Exhibits Combination Benefit with AZD4573 In Vitro and In Vivo

### Methods

The BTK inhibitor-sensitive ABC-DLBCL cell line OCILy10 was treated in vitro for an 8-hour time course with either 100 nM AZD4573 alone or following a 24-hour pre-treatment with 30 nM ibrutinib since, similar to acalabrutinib, exposure for 24 hours was sufficient to induce maximal BH3-only protein levels (data not shown). Cells were harvested for protein lysates at varying time points (0, 0.5, 1, 2, 4, and 8 hours) post-AZD4573 treatment, normalized for protein concentration using the BCA Protein Assay Kit, and run for Western blots according to standard protocols. To ensure expected target engagement was achieved, the blots were probed for the proximal and distal biomarkers for CDK9 (pSer2-RNAPolII and Mcl-1, respectively) and the biomarker for activated BTK (pBTK). The BH3-only proteins Bim and Bmf were also probed to confirm increased levels. To gauge the time to induction of apoptosis, cleaved caspase-3 was also assessed. A loading control (vinculin) was also utilized for normalization.

AZD4573 was formulated in dimethylacetamide (DMA)/polyethylene glycol 400 (PEG 400)/1% w/v Tween 80 solution 2/30/68 and dosed at 5 mL/kg, intraperitoneally (ip), bi-daily (BID) with a 2 hour split on days 1 and 2 with a 5 day dose holiday. Ibrutinib was formulated in 0.5% hydroxypropyl methyl cellulose, and dosed once daily (QD) as an oral (po) administration at a volume of 10 ml/kg at a dose of 12 mg/kg. 5×10⁶ OCI-Ly10 tumor cells were injected subcutaneously in the right flank of C.B.-17 SCID female mice in a volume of 0.1 mL containing 50% matrigel. Tumor volumes (measured by caliper), animal body weight, and tumor condition were recorded twice weekly for the duration of the studies. The tumor volume was calculated using the formula: length (mm)×width (mm)²×0.52. For efficacy studies, growth inhibition from the start of treatment was assessed by comparison of the differences in tumor volume between control and treated groups. Dosing began when mean tumor size reached approximately 150-180 mm³. CR=complete response.

### Discussion

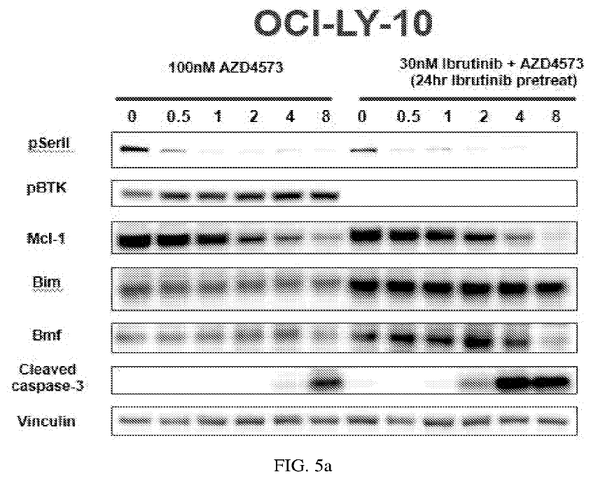

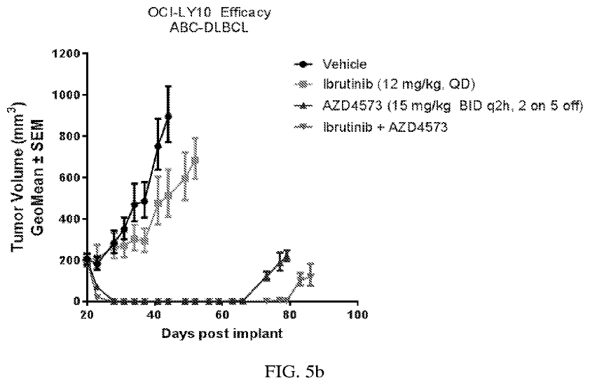

Like acalabrutinib, ibrutinib increased levels of the BH3-only proteins Bim and Bmf in the BTK inhibitor-sensitive model OCILy10, consistent with BTK inhibition (FIG. 5a). Adding AZD4573 following a 24 hour pre-treatment with ibrutinib resulted in enhanced and rapid induction of caspase (FIG. 5a), which translated to improved duration of response in vivo (FIG. 5b). Twice weekly administration of AZD4573 yielded complete response in 100% of treated animals following 3 cycles of dosing, compared with ibrutinib single agent which was not highly effective. Combination of AZD4573 with ibrutinib also yielded complete responses in 100% of treated animals following 3 cycles of treatment. AZD4573 in combination with ibrutinib yielded a two week delay to tumor regrowth in OCILy10, compared with single agent AZD4573 treatment, demonstrating an increased duration of response for the combination. All therapies were well tolerated whether dosed alone or in combination.

Other embodiments are within the scope of the following claims.

1-7. (canceled)

8. A method of treating cancer in a subject, comprising:
administering an effective amount of a BTK inhibitor to the subject; and
administering an effective amount of an inhibitor of CDK9 to the subject, wherein the BTK inhibitor is acalabrutinib and the inhibitor of CDK9 is AZD4573.

9. The method of claim 8, wherein acalabrutinib is administered to the subject before AZD4573 is administered to the subject.

10-13. (canceled)

14. The method claim 8, wherein the cancer is selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.

15. A kit comprising:
a first pharmaceutical composition comprising acalabrutinib and a pharmaceutically acceptable carrier; and
a second pharmaceutical composition comprising AZD4573 and a pharmaceutically acceptable carrier.

16. The method claim 9, wherein the cancer is selected from diffuse large B-cell lymphoma, mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia.
